(19) United States
(12) Patent Application Publication
(10) Pub. No.: US 2024/0160299 A1
(43) Pub. Date: May 16, 2024

(54) AN ELECTRONIC INPUT WRITING DEVICE FOR DIGITAL CREATION AND A METHOD FOR OPERATING THE SAME

(71) Applicant: TOOLIQA INNOVATIONS LLP, GURGAON (IN)

(72) Inventor: ADITYA RAJ, GURGAON (IN)

(21) Appl. No.: 17/776,614

(22) PCT Filed: Jun. 11, 2021
(86) PCT No.: PCT/IB2021/055149

§ 371 Date: May 13, 2022

(30) Foreign Application Priority Data
Apr. 28, 2021 (IN) 202111019487

(51) Int. Cl. G06F 3/0354; G01K 1/024; G01K 3/00; G01K 13/20; G06F 3/0338; G06F 3/039; G06V 10/56; G06V 10/94; G06V 30/142; G06V 30/19; G10L 15/18; G10L 15/22

(52) U.S. Cl. CPC G06F 3/03542; G01K 1/024; G01K 3/005; G01K 13/20; G06F 3/0338; G06F 3/0393; G06V 10/56; G06V 10/95; G06V 30/1423; G06V 30/19; G10L 15/18; G10L 15/22; G10L 2015/223

(57) ABSTRACT

An electronic writing device is disclosed. The device includes an inertial measurement unit to measure motion of a finger of a user, activates the electronic writing device to perform pincer grip analysis of the user. An image acquisition unit captures one or more images of an object present. A colour sensor to recognize one or more colours of the one or more images of the object captured. A light sensor illuminates a colour of light corresponding to the one or more colours of the one or more images of the object. An image processing subsystem creates a multi-dimensional representation of the one or more images of the object, identifies one or more parameters associated with the object from the multi-dimensional representation created, recognizes a plurality of characters from the multi-dimensional representation of the one or more images of the object, analyzes a language of the plurality of characters recognized from the multi-dimensional representation.

### CROSS-REFERENCE TO RELATED APPLICATION

This National Phase Application claims priority from a complete patent application filed in India having Patent Application No. 202111019487, filed on Apr. 28, 2021 and titled “AN ELECTRONIC INPUT WRITING DEVICE FOR DIGITAL CREATION AND A METHOD FOR OPERATING THE SAME”.

### FIELD OF INVENTION

Embodiments of a present disclosure relate to an electronic input device and more particularly to, an electronic input writing device for digital creation and a method for operating the same.

### BACKGROUND

An electronic input writing device is an electronic input device that digitally captures writing gestures of a user and converts the captured gestures to digital information which may be utilized in a variety of applications. There are several electronic writing devices for entering data into a computing device such as keyboard, styluses and pen.

Furthermore, pen-based digital devices have been introduced, for capturing the gestures and converting the same to digital information, which are useful, portable and greatly desired. The user of such devices may often desire to share the pen-based digital device with others. With advancement in technology, some pen based digital devices have been introduced which captures a handwriting of the user and converts a handwritten information into digital data. However, these devices do not produce an accurate recording of the text or graphics that have been input via the writing surface. Considerable information indicative of the motion of the pen is lost in the processing of data. One reason is that data describing the motion of the pen is under sampled.

Also, existing devices utilize accelerometer, gyroscope and magnetometer to determine the position of the writing device on the physical surface. The accelerometer and gyroscope are being used to provide a frame of reference to the position information which is collected by the accelerometer. However, use of positional sensor along with the accelerometer and the gyroscope results in increase in cost of the writing device. Moreover, few writing devices exist which calculate the position of the writing device using the acceleration received from the accelerometer. By double integrating the acceleration and using the high frequency noise from the accelerometer, the position of the writing device on the physical surface may be determined. However, in such devices, constants of integration results in large DC errors.

As information technology (IT) penetrates all commercial and public transactions and communications, it is important to ensure accessibility to everyone. Many countries have begun to define new regulations and standards to enable people with disabilities to easily access information technology. Currently available assistance for blind and visually impaired people comprises a wide range of technical solutions, including document scanners and enlargers, interactive speech software and cognitive tools, screen reader software and screen enlargement programs. However, such solutions suffer from a variety of functional and operational deficiencies that limit their usefulness.

Hence, there is a need for an improved electronic input writing device for digital creation to address the aforementioned issue(s).

### BRIEF DESCRIPTION

In accordance with an embodiment of the present disclosure, an electronic input writing device for digital creation is disclosed. The device includes an electronic writing module. The electronic writing module includes an inertial measurement unit configured to measure motion of a finger of a user by receiving a tactile input. The inertial measurement unit is also configured to activate the electronic writing device to perform pincer grip analysis of the user based on the motion of the finger of the user measured. The electronic writing module also includes an image acquisition unit located in proximity to a tip of the electronic writing device. The image acquisition unit is configured to calculate distance from an object of interest present in an environment by emitting infra-red ray upon activation of the electronic writing device. The image acquisition unit is also configured to capture one or more images of the object present in the environment based on the distance calculated. The electronic writing module also includes a colour sensor operatively coupled to the image acquisition unit. The colour sensor is configured to recognize one or more colours of the one or more images of the object captured. The electronic writing module also includes a light sensor encircled on the electronic writing device. The light sensor illuminates a colour of light corresponding to the one or more colours of the one or more images of the object captured. The device also includes an image processing subsystem hosted on a server and communicatively coupled to the electronic writing module. The image processing subsystem is configured to create a multi-dimensional representation of the one or more images of the object captured using a photogrammetry process. The image processing subsystem is also configured to identify one or more parameters associated with the object from the multi-dimensional representation created of the one or more images of the object using a learning technique. The image processing subsystem is also configured to recognize a plurality of characters from the multi-dimensional representation of the one or more images of the object created using an optical character recognition technique. The image processing subsystem is also configured to analyze a language of the plurality of characters recognized from the multi-dimensional representation for utilization by the user using a natural language processing technique. The device also includes an object collaboration subsystem communicatively coupled to the image processing subsystem. The object collaboration subsystem is configured to obtain the one or more images captured by each corresponding electronic writing module associated with a plurality of users. The object collaboration subsystem is configured to establish a communication link with the server for collaborating the one or more images obtained from each of the corresponding electronic writing module. The object collaboration subsystem is also configured to connect with a plurality of external computing devices, upon collaboration, for displaying the one or more images of the object captured on a screen of the plurality of external computing devices associated with the plurality of users.

In accordance with another embodiment of the present disclosure, a method for operating an electronic input writing device for digital creation is disclosed. The method includes measuring, by an inertial measurement unit, motion of a finger of a user by receiving a tactile input. The method also includes activating, by the inertial measurement unit, the electronic writing device to perform pincer grip analysis of the user based on the motion of the finger of the user measured. The method also includes calculating, by an image acquisition unit, distance from an object of interest present in an environment by emitting infra-red ray upon activation of the electronic writing device. The method also includes capturing, by the image acquisition unit, one or more images of the object present in the environment based on the distance calculated. The method also includes recognizing, by a colour sensor, one or more colours of the one or more images of the object captured. The method also includes illuminating, by a light sensor, a colour of light corresponding to the one or more colours of the one or more images of the object captured. The method also includes creating, by an image processing subsystem, a multi-dimensional representation of the one or more images of the object captured using a photogrammetry process. The method also includes identifying, by the image processing subsystem, one or more parameters associated with the object from the multi-dimensional representation created of the one or more images of the object using a learning technique. The method also includes recognizing, by the image processing subsystem, a plurality of characters from the multi-dimensional representation of the one or more images of the object created using an optical character recognition technique. The method also includes analyzing, by the image processing subsystem, a language of the plurality of characters recognized from the multi-dimensional representation for utilization by the user using a natural language processing technique. The method also includes obtaining, by an object collaboration subsystem, the one or more images captured by each corresponding electronic writing module associated with a plurality of users. The method also includes establishing, by the object collaboration subsystem, a communication link with the server for collaborating the one or more images obtained from each of the corresponding electronic writing module. The method also includes connecting, by the object collaboration subsystem, with a plurality of external computing devices, upon collaboration, for displaying the one or more images of the object captured on a screen of the plurality of external computing devices associated with the plurality of users.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF THE DRAWINGS

The disclosure will be described and explained with additional specificity and detail with the accompanying figures in which:

FIG. 1 is a block diagram representation of an electronic input writing device for digital creation in accordance with an embodiment of the present disclosure;

FIG. 2 illustrates a schematic representation of an embodiment of an electronic input writing device for digital creation of FIG. 1 in accordance with an embodiment of the present disclosure; and

FIG. 3(a) and FIG. 3(b) is a flow chart representing the steps involved in a method for operation of an electronic input writing device for digital creation of FIG. 1 in accordance with the embodiment of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure.

The terms “comprises”, “comprising”, or any other variations thereof, are intended to cover a non-exclusive inclusion, such that a process or method that comprises a list of steps does not include only those steps but may include other steps not expressly listed or inherent to such a process or method. Similarly, one or more devices or sub-systems or elements or structures or components preceded by “comprises . . . a” does not, without more constraints, preclude the existence of other devices, sub-systems, elements, structures, components, additional devices, additional sub-systems, additional elements, additional structures or additional components. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

In the following specification and the claims, reference will be made to a number of terms, which shall be defined to have the following meanings. The singular forms “a”, “an”, and “the” include plural references unless the context clearly dictates otherwise.

Embodiments of the present disclosure relate to a device and a method for operating an electronic input writing device for digital creation. The device includes an electronic writing module. The electronic writing module includes an inertial measurement unit configured to measure motion of a finger of a user by receiving a tactile input. The inertial measurement unit is also configured to activate the electronic writing device to perform pincer grip analysis of the user based on the motion of the finger of the user measured. The electronic writing module also includes an image acquisition unit located in proximity to a tip of the electronic writing device. The image acquisition unit is configured to calculate distance from an object of interest present in an environment by emitting infra-red ray upon activation of the electronic writing device. The image acquisition unit is also configured to capture one or more images of the object present in the environment based on the distance calculated. The electronic writing module also includes a colour sensor operatively coupled to the image acquisition unit. The colour sensor is configured to recognize one or more colours of the one or more images of the object captured. The electronic writing module also includes a light sensor encircled on the electronic writing device. The light sensor illuminates a colour of light corresponding to the one or more colours of the one or more images of the object captured. The device also includes an image processing subsystem hosted on a server. The image processing subsystem is configured to create a multi-dimensional representation of the one or more images of the object captured using a photogrammetry process. The image processing subsystem is also configured to identify one or more parameters associated with the object from the multi-dimensional representation created of the one or more images of the object using a learning technique. The image processing subsystem is also configured to recognize a plurality of characters from the multi-dimensional representation of the one or more images of the object created using an optical character recognition technique. The image processing subsystem is also configured to analyze a language of the plurality of characters recognized from the multi-dimensional representation for utilization by the user using a natural language processing technique. The device also includes an object collaboration subsystem communicatively coupled to the image processing subsystem. The object collaboration subsystem is configured to obtain the one or more images captured by each corresponding electronic writing module associated with a plurality of users. The object collaboration subsystem is configured to establish a communication link with the server for collaborating the one or more images obtained from each of the corresponding electronic writing module. The object collaboration subsystem is also configured to connect with a plurality of external computing devices, upon collaboration, for displaying the one or more images of the object captured on a screen of the plurality of external computing devices associated with the plurality of users.

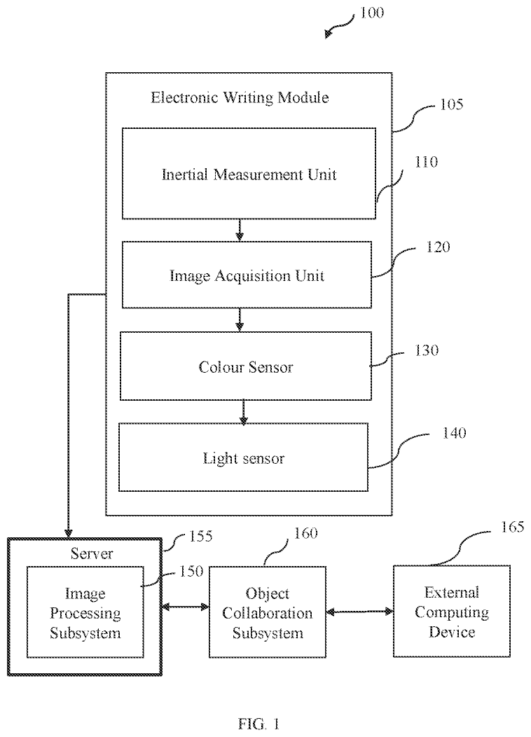

FIG. 1 is a block diagram representation of an electronic input writing device 100 for digital creation in accordance with an embodiment of the present disclosure. The electronic input writing device 100 is combination of both software components as well as the hardware components. The electronic input writing device 100 includes an electronic writing module 105. As used herein, the term ‘electronic writing module’ is defined as a technologically advanced device which captures elements that is utilized further for writing or drawing for one or more digital creations. In one embodiment, the electronic writing module 105 may include, but not limited to, a stylus, a pen, a pencil, a crayon and the like. The electronic writing module 105 includes an inertial measurement unit (IMU) 110 configured to measure motion of a finger of a user by receiving a tactile input. In one embodiment, the IMU includes an accelerometer, a gyroscope and a magnetometer. In some embodiment, the tactile input may include, but not limited to, touch, pressure, vibration and the like. The inertial measurement unit is also configured to activate the electronic writing device to perform pincer grip analysis of the user based on the motion of the finger of the user measured. The IMU 110 also performs handwriting review based on the pincer grip analysis. As used herein, the term ‘pincer grip analysis’ is defined as an analysis done on an action of closing the thumb and index finger together by a user in order to hold an object.

The electronic writing module 105 also includes an image acquisition unit 120 located in proximity to a tip 115 of the electronic writing device 100. In one embodiment, the image acquisition unit 120 may include at least one of a camera, an optical sensor, an infrared sensor (IR) or a combination thereof. The image acquisition unit 120 is configured to calculate distance from an object of interest present in an environment by emitting infra-red ray using the IR sensor upon activation of the electronic writing device. The image acquisition unit 120 is also configured to capture one or more images of the object present in the environment based on the distance calculated. The electronic writing module 105 is equipped with one or more powerful cameras enables to capture real world objects in high definition 2D or 3D image format.

The electronic writing module 105 also includes a colour sensor 130 operatively coupled to the image acquisition unit 120. The colour sensor 130 is configured to recognize one or more colours of the one or more images of the object captured. In one embodiment, the colour sensor may include a red, green blue (RGB) sensor. In such embodiment, the colour sensor is configured to record one or more red, green blue (RGB) pixel values of the one or more images of the object. This device 100 enables the user to capture any object or subject around them in the environment or the nature in its true 3d form and along with its true colours such as RGB or cyan, magenta, yellow, black (CMYK) or hexadecimal (Hex) values.

The electronic writing module 105 also includes a light sensor 140 encircled on the electronic writing device. The light sensor 140 illuminates a colour of light corresponding to the one or more colours of the one or more images of the object captured. In one embodiment, the light sensor 140 may include a light emitting diode (LED) sensor. In such embodiment, light sensor is arranged on the electronic device as a ring.

The device 100 also includes an image processing subsystem 150 hosted on a server 155. In one embodiment, the server may include a remote server. In such embodiment, the remote server may include a cloud server. In another embodiment, the server may include a local server. The image processing subsystem 150 is configured to create a multi-dimensional representation of the one or more images of the object captured using a photogrammetry process. In a specific embodiment, the image processing subsystem also produces a customized colour palette for utilization in a design application by picking the one or more colours of the one or more images of the object being recognized. The image processing subsystem 150 finds the RGB values for each of the pixels and store unique image colours in the customized colour palette of the design application. In such embodiment, the design application may include Adobe Suite™, MS Paint™, computer aided design (CAD) software and the like.

The image processing subsystem 150 is also configured to identify one or more parameters associated with the object from the multi-dimensional representation created of the one or more images of the object using a learning technique. As used herein, the term ‘learning technique’ is defined as a machine learning technique which makes the system self-sufficient in identifying several parameters associated with the object without being explicitly programmed. In a specific embodiment, the learning technique may include, but not limited to, a fast convolutional neural network (F-CNN), a histogram of oriented gradients (HOG), a single shot detector (SSD), a region based fully convolutional network (R-FCN), you only look once (YOLO) and the like. In one embodiment, the object learning technique may also include object segmentation such as mask region based convolutional neural network (RCNN) and object reconstruction technique such as generative adversarial network (GAN) and the like. In one embodiment, the one or more parameters associated with the object may include at least one of a shape of the object, a size of the object, a pattern of the object, a text present in the one or more images of the object or a combination thereof.

The image processing subsystem 150 is also configured to recognize a plurality of characters from the multi-dimensional representation of the one or more images of the object created using an optical character recognition (OCR) technique. The OCR technique scans the multi-dimensional representation of the one or more images of the object to recognize the plurality of characters. Once, the plurality of characters are recognized, such plurality of characters are converted into digital format. The image processing subsystem 150 is also configured to analyze a language of the plurality of characters recognized from the multi-dimensional representation for utilization by the user using a natural language processing (NLP) technique. The electronic writing module 105 assists one or more visually impaired/challenged users as well as normal users with reading inability by scanning text from the focused document through the OCR technique and further interpreting the language through the NLP technique in order to read it loud in multiple other languages of user's choice. The user further stores the captured text on their corresponding cloud storage such as a drive for future reading/listening through simple voice commands. In a particular embodiment, the image processing subsystem also detects an inappropriate content searched or accessed by the user through implementation of a profanity and obscene filter. While offering internet connectivity, the device has inbuilt controls to share filtered information with the user which is age appropriate. For example, if the user is a child, then a parent email ID is required during device's initial setup so that the inappropriate contents searched by the user can be monitored by the parent.

The device 100 also includes an object collaboration subsystem 160 communicatively coupled to the image processing subsystem 150. The object collaboration subsystem 160 obtains the one or more images captured by each corresponding electronic writing module associated with a plurality of users. The object collaboration subsystem 160 is configured to establish a communication link with the server for collaborating the one or more images obtained from each of the corresponding electronic writing module. The object collaboration subsystem 160 is also configured to connect with a plurality of external computing devices, upon collaboration, for displaying the one or more images of the object captured on a screen of the plurality of external computing devices associated with the plurality of users. In one embodiment, the plurality of external computing devices may include, but not limited to, a personal digital assistant (PDA), a tablet, a laptop, a desktop, a smartphone, a smart watch and the like. The device 100 establishes the communication link via at least one of Bluetooth network, a Wi-Fi network, a long-term evaluation (LTE) network or a combination thereof. The device 100 upon establishing the communication with the plurality of external computing devices 165 also provides drag and drop, auto syncing experience for visualising multi-dimensional representation of the one or more images on the connected computing device from the cloud server.

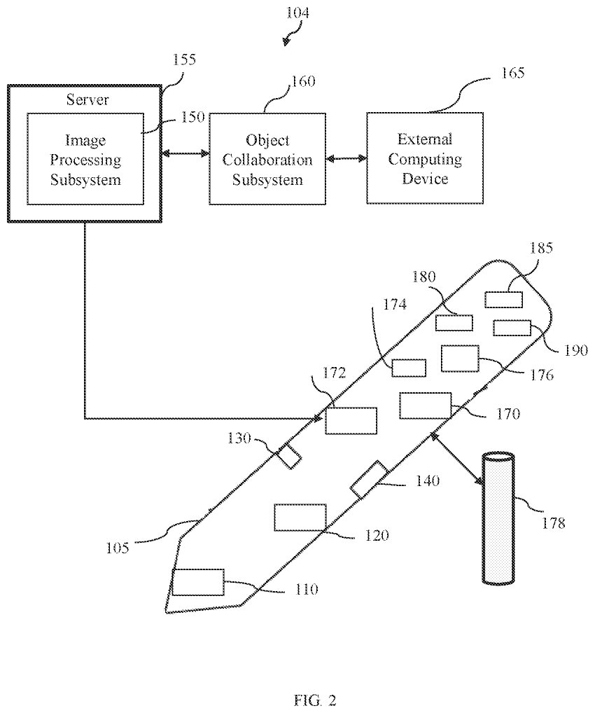

FIG. 2 illustrates a schematic representation 104 of an embodiment of an electronic input writing device 100 for digital creation of FIG. 1 in accordance with an embodiment of the present disclosure. As described in aforementioned FIG. 1, the device 100 includes an electronic writing module 105 which includes an inertial measurement unit (IMU) 110, an image acquisition unit 120, a colour sensor 130, a light sensor 140, an image processing subsystem 150 and an interfacing subsystem 160. In addition, the device 100 also includes an interactive digital assistant 170 configured to receive a plurality of commands from the user in a voice format for performing one or more operations. An NLP based interactive digital assistant takes command from the user to switch modes to perform various operations such as 2D or 3D image capture, explore surrounding objects using image search feature, picks colour of the surrounding object for use and storing it in palette, answering questions about weather, date or general knowledge, transferring or deleting photos, posting photos on social media using connected account and the like. The device 100 also has inbuilt memory storage 172, battery 174 and microprocessor 176 for mobility and intelligence. For recharging the battery 174, the device 100 also includes a docking wireless charging stand 178 to support charging of the electronic writing device.

Further, the device 100 also includes one or more wireless connection enabled speakers 180 configured to generate a voice output representative one or more contents associated with the object. The device 100 also includes a joystick sensor 185 configured to enable the user to navigate on a screen of the external computing device for interaction with on-screen objects, wherein the external computing device is connected with the electronic writing device. The device 100 also includes a thermal sensor 190 configured to detect body temperature of the user based on the tactile input received. The thermal sensor 190 is also configured to generate an alarm signal when the body temperature of the user deviates from a predetermined threshold value. As used herein, the term ‘predetermined threshold value’ is defined as a temperature value or a limit which is set corresponding to market standard. In one embodiment, the alarm signal is raised via an email, call, message and the like when body temperature of the user rises or falls beyond the predetermined threshold value.

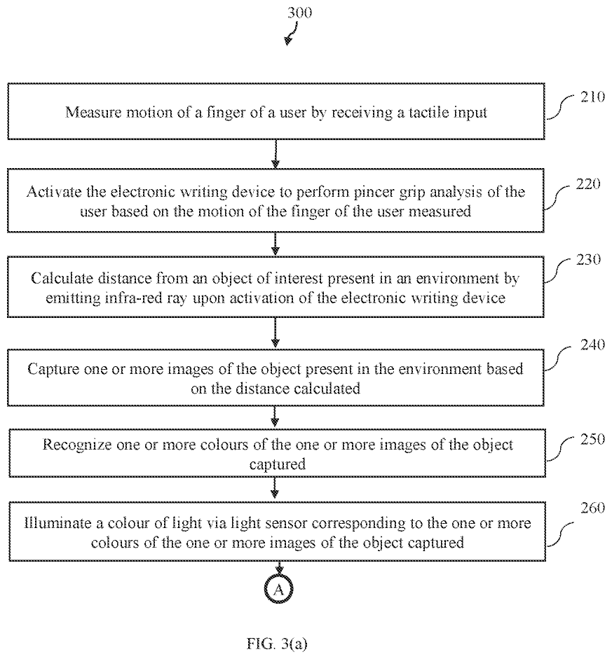

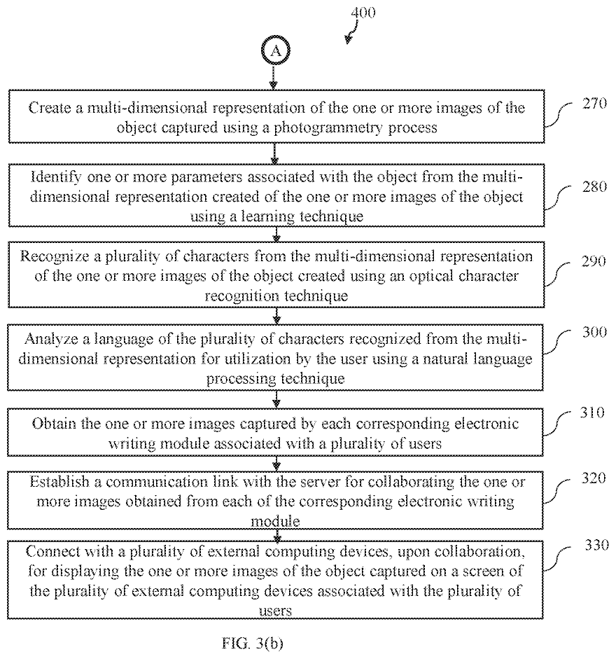

FIG. 3(a) and FIG. 3(b) is a flow chart representing the steps involved in a method 200 for operation of an electronic input writing device for digital creation of FIG. 1 in accordance with the embodiment of the present disclosure. The method 200 includes measuring, by an inertial measurement unit, motion of a finger of a user by receiving a tactile input in step 210. In one embodiment, measuring the motion of the finger of the user may include measuring the motion of the finger of the user by receiving the tactile input including, but not limited to, touch, pressure, vibration and the like. The method 200 also includes activating, by the inertial measurement unit, the electronic writing device to perform pincer grip analysis of the user based on the motion of the finger of the user measured in step 220. In one embodiment, performing the pincer grip analysis of the user based on the motion of the finger of the user may include performing the pincer grip analysis by at least a stylus, a pen, a pencil, a crayon and the like.

The method 200 also includes calculating, by an image acquisition unit, distance from an object of interest present in an environment by emitting infra-red ray upon activation of the electronic writing device in step 230. The method 200 also includes capturing, by the image acquisition unit, one or more images of the object present in the environment based on the distance calculated in step 240. In one embodiment, capturing the one or more images of the object present in the environment may include capturing the one or more images by at least one of a camera, an optical sensor, an infrared sensor (IR) or a combination thereof.

The method 200 also includes recognizing, by a colour sensor, one or more colours of the one or more images of the object captured in step 250. In some embodiment, recognizing the one or more colours of the one or more images of the object may include recognizing the one or more colours of the object by using a red, green, blue (RGB) sensor. In such embodiment, the colour sensor records one or more red, green blue (RGB) pixel values of the one or more images of the object. In another embodiment, the colour sensor may also record cyan, magenta, yellow, black (CMYK) values or hexadecimal (Hex) values of the one or more images of the object.

The method 200 also includes illuminating, by a light sensor, a colour of light corresponding to the one or more colours of the one or more images of the object captured in step 260. In one embodiment, illuminating the colour of the light corresponding to the one or more colours of the one or more images of the object may include illuminating the colour of the light in a form of a light emitting diode (LED) sensor. In such embodiment, the LED sensor is coupled to the electronic writing device in a form of a ring. The method 200 also includes creating, by an image processing subsystem, a multi-dimensional representation of the one or more images of the object captured using a photogrammetry process in step 270.

The method 200 also includes identifying, by the image processing subsystem, one or more parameters associated with the object from the multi-dimensional representation created of the one or more images of the object using a learning technique in step 280. In one embodiment, identifying the one or more parameters associated with the object from the multi-dimensional representation created may include identifying at least one of a shape of the object, a size of the object, a pattern of the object, a text present in the one or more images of the object or a combination thereof. In some embodiment, identifying the one or more parameters associated with the object from the multi-dimensional representation created may include identifying the one or more parameters using at least one of a fast convolutional neural network (F-CNN), a histogram of oriented gradients (HOG), a single shot detector (SSD), a region based fully convolutional network (R-FCN), you only look once (YOLO) or a combination thereof. In one embodiment, the object learning technique may also include object segmentation such as mask region based convolutional neural network (RCNN) and object reconstruction technique such as generative adversarial network (GAN) and the like.

The method 200 also includes recognizing, by the image processing subsystem, a plurality of characters from the multi-dimensional representation of the one or more images of the object created using an optical character recognition technique in step 290. In one embodiment, recognizing the plurality of characters from the multi-dimensional representation of the one or more images of the object may include scanning the multi-dimensional representation of the one or more images of the object to recognize the plurality of characters. Once, the plurality of characters are recognized, such plurality of characters are converted into digital format. The method 200 also includes analyzing, by the image processing subsystem, a language of the plurality of characters recognized from the multi-dimensional representation for utilization by the user using a natural language processing technique in step 300.

The method 200 also includes obtaining, by an object collaboration subsystem, the one or more images captured by each corresponding electronic writing module associated with a plurality of users in step 310. The method 200 also includes establishing, by the object collaboration subsystem, a communication link with the server for collaborating the one or more images obtained from each of the corresponding electronic writing module in step 320. The method 200 also includes connecting, by the object collaboration subsystem, with a plurality of external computing devices, upon collaboration, for displaying the one or more images of the object captured on a screen of the plurality of external computing devices associated with the plurality of users in step 330. In one embodiment, establishing the communication link with the plurality of external computing devices for displaying the one or more images may include establishing the communication link via a Bluetooth, Wi-Fi, LTE network and the like. In such embodiment, the external computing device may include, but not limited to, a personal digital assistant (PDA), a tablet, a laptop, a desktop, a smartphone, a smart watch and the like.

Various embodiments of the present disclosure provides an intuitive, user friendly device that equips one to explore the world and capture the elements and objects in it's a real form for their digital creations.

Moreover, the present disclosed device is easy and comfortable to work with which puts all the colours of the world in hands of the user. The device is capable of scanning any colour and starts drawing or writing with it instantly. Not only, but this the device also stores the colours too, which enables the user to upload, share and use them wherever, whenever they want based on requirement.

It will be understood by those skilled in the art that the foregoing general description and the following detailed description are exemplary and explanatory of the disclosure and are not intended to be restrictive thereof.

While specific language has been used to describe the disclosure, any limitations arising on account of the same are not intended. As would be apparent to a person skilled in the art, various working modifications may be made to the method in order to implement the inventive concept as taught herein.

The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, the order of processes described herein may be changed and are not limited to the manner described herein. Moreover, the actions of any flow diagram need not be implemented in the order shown; nor do all of the acts need to be necessarily performed. Also, those acts that are not dependent on other acts may be performed in parallel with the other acts. The scope of embodiments is by no means limited by these specific examples.

We claim:

1. An electronic input writing device for digital creation comprising:
an electronic writing module comprising:
an inertial measurement unit configured to:
measure motion of a finger of a user by receiving a tactile input; and
activate the electronic writing device to perform pincer grip analysis of the user based on the motion of the finger of the user measured;
an image acquisition unit located in proximity to a tip of the electronic writing device wherein the image acquisition unit is configured to:
calculate distance from an object of interest present in an environment by emitting infra-red ray upon activation of the electronic writing device; and
capture one or more images of the object present in the environment based on the distance calculated;
a colour sensor operatively coupled to the image acquisition unit, wherein the colour sensor is configured to recognize one or more colours of the one or more images of the object captured;
a light sensor encircled on the electronic writing module, wherein the light sensor illuminates a colour of light corresponding to the one or more colours of the one or more images of the object captured;
an image processing subsystem hosted on a server and communicatively coupled to the electronic writing module, wherein the image processing subsystem is configured to:
create a multi-dimensional representation of the one or more images of the object captured using a photogrammetry process;
identify one or more parameters associated with the object from the multi-dimensional representation created of the one or more images of the object using a learning technique;
recognize a plurality of characters from the multi-dimensional representation of the one or more images of the object created using an optical character recognition technique; and
analyze a language of the plurality of characters recognized from the multi-dimensional representation for utilization by the user using a natural language processing technique; and
an object collaboration subsystem operatively coupled to the image processing subsystem, wherein the object collaboration subsystem is configured to:
obtain the one or more images captured by each corresponding electronic writing module associated with a plurality of users;
establish a communication link with the server for collaborating the one or more images obtained from each of the corresponding electronic writing module;
connect with a plurality of external computing devices, upon collaboration, for displaying the one or more images of the object captured on a screen of the plurality of external computing devices associated with the plurality of users.

2. The device as claimed in claim 1, wherein the image acquisition unit comprises at least one of a camera, an optical sensor, an infrared sensor or a combination thereof.

3. The device as claimed in claim 1, wherein the colour sensor comprises a red, green blue sensor, wherein the colour sensor is configured to record one or more red, green blue pixel values of the one or more images of the object.

4. The device as claimed in claim 1, wherein the image processing subsystem is configured to produce a customized colour palette for utilization in a design application by picking the one or more colours of the one or more images of the object being recognized.

5. The device as claimed in claim 1, wherein the image processing subsystem is configured to detect an inappropriate content searched or accessed by the user through implementation of a profanity and obscene filter.

6. The device as claimed in claim 1, wherein the one or more parameters associated with the object comprises at least one of a shape of the object, a size of the object, a pattern of the object, a text present in the one or more images of the object or a combination thereof.

7. The device as claimed in claim 1, comprising an interactive digital assistant configured to receive a plurality of commands from the user in a voice format for performing one or more operations.

8. The device as claimed in claim 1, comprising one or more wireless connection enabled speakers configured to generate a voice output representative one or more contents associated with the object.

9. The device as claimed in claim 1, comprising a joystick sensor configured to enable the user to navigate on a screen of the external computing device for interaction with on-screen objects, wherein the external computing device is connected with the electronic writing device.

10. The device as claimed in claim 1, comprising a docking wireless charging stand to support charging of a battery of the electronic writing device.

11. The device as claimed in claim 1, comprising a thermal sensor configured to:
detect body temperature of the user based on the tactile input received; and
generate an alarm signal when the body temperature of the user deviates from a predetermined threshold value.

12. A method for operating an electronic writing device comprising:
measuring, by an inertial measurement unit, motion of a finger of a user by receiving a tactile input;
activating, by the inertial measurement unit, the electronic writing device to perform pincer grip analysis of the user based on the motion of the finger of the user measured;
calculating, by an image acquisition unit, distance from an object of interest present in an environment by emitting infra-red ray upon activation of the electronic writing device;
capturing, by the image acquisition unit, one or more images of the object present in the environment based on the distance calculated;
recognizing, by a colour sensor, one or more colours of the one or more images of the object captured;
illuminating, by a light sensor, a colour of light corresponding to the one or more colours of the one or more images of the object captured;
creating, by an image processing subsystem, a multi-dimensional representation of the one or more images of the object captured using a photogrammetry process;
identifying, by the image processing subsystem, one or more parameters associated with the object from the multi-dimensional representation created of the one or more images of the object using a learning technique;
recognizing, by the image processing subsystem, a plurality of characters from the multi-dimensional representation of the one or more images of the object created using an optical character recognition technique;
analyzing, by the image processing subsystem, a language of the plurality of characters recognized from the multi-dimensional representation for utilization by the user using a natural language processing technique;
obtaining, by an object collaboration subsystem, the one or more images captured by each corresponding electronic writing module associated with a plurality of users;
establishing, by the object collaboration subsystem, a communication link with the server for collaborating the one or more images obtained from each of the corresponding electronic writing module; and
connecting, by the object collaboration subsystem, with a plurality of external computing devices, upon collaboration, for displaying the one or more images of the object captured on a screen of the plurality of external computing devices associated with the plurality of users.
